(19) United States
(12) Patent Application Publication
(10) Pub. No.: US 2024/0237971 A1
(43) Pub. Date: Jul. 18, 2024

(54) SAMPLE COLLECTION DEVICE AND SYSTEM

(71) Applicant: 3M INNOVATIVE PROPERTIES COMPANY, St. Paul, MN (US)

(72) Inventors: Michael R. Berrigan, Oakdale, MN (US); Tonya D. Bonilla, Woodbury, MN (US); Alan R. Dombrowski, Woodbury, MN (US); Laura R. Nereng, Woodbury, MN (US); Audrey A. Sherman, Woodbury, MN (US); Brett J. Sitter, Marine on St. Croix, MN (US); Narina Y. Stepanova, Woodbury, MN (US); Michael C. Wohl, Bethesda, MD (US)

(21) Appl. No.: 18/562,757

(22) PCT Filed: Feb. 11, 2022
(86) PCT No.: PCT/IB2022/051251

§ 371 Date: Nov. 20, 2023

Related U.S. Application Data
(60) Provisional application No. 63/201,983, filed on May 21, 2021.
(60) Provisional application No. 63/227,529, filed on Jul. 30, 2021.
(60) Provisional application No. 63/260,828, filed on Sep. 1, 2021.

(51) Int. Cl. A61B 10/00

(52) U.S. Cl. CPC A61B 10/00; A61B 2010/0006; A61B 2010/0087

(57) ABSTRACT

A sample collection device includes a housing extending from a mouthpiece end to an air outlet end, the housing defining an airflow channel from the mouthpiece end to the air outlet end, the mouthpiece end configured to receive an exhalation airflow. A porous sample collection media is fixed within the housing and along the airflow channel. A mouthpiece end cap is replaceably coupled to the mouthpiece end and an air outlet end cap is replaceably coupled to the air outlet end.

### BACKGROUND

As Covid-19 reached pandemic status, increased availability of diagnostic testing was important to help identify and control the serious illness. This illness has highlighted the need for widespread availability of such diagnostic tests even after the pandemic ends. Diagnostic tests used to test for the presence of a virus or other pathogen in the airways, throat, or nasopharynx typically involve the insertion of a swab into the back of the nasal passage, the mid-turbinate area of the nasal passage, the anterior nares, or the throat to obtain a sample. The swab is then inserted into a container and analyzed or sent to a lab for processing. Other diagnostic tests involve collecting a saliva sample and then placing it in a container. Currently available at—home viral tests (e.g., COVID-19 tests) involve a nasal swab and a test kit (for example, the Ellume™ test, the Abbot™ BinaxNOW™ test, and the Lucira™ All-in-One test kit). Tests that utilize nasal swab samples or saliva contend with contaminants that can interfere with the various diagnostic tests. As a result, these sample types require a purification step when using RT-PCR molecular testing.

### SUMMARY

The sample collection devices and test processes described above have various challenges. For example, the available tests typically require that the collection device be processed at a laboratory, increasing cost and delaying delivery of results. Further, many of the test methods require that the sample collection mechanism be a nasopharyngeal or other type of nasal or oral swab, which is uncomfortable for the user. This discomfort can cause users to opt out of testing. Further, there may be possibility of contamination of the sample during transfer to the clean container, removal from the container, etc. Due to the multiple steps and devices involved and the possibility of contamination of the sample, such conventional methods and devices for sample collection and eluent testing may be used by only trained professionals (e.g., medical personnel), and may be complicated for use by a user with little or no training.

In some embodiments, the present disclosure provides easy-to-use, inexpensive integrated sample collection and testing devices in which sample collection and sample testing happen in a single integrated device that can be used in any location by a layperson. Other embodiments of the present disclosure provide a sample collection device that does not require the user to undergo a nasopharyngeal or other nasal or oral swab. In some embodiments, the integrated sample collection and testing devices and/or sample collection device collects only bioaerosol. The bio-aerosol can be, for example, from nasal or oral exhalation.

In some embodiments, the integrated sample collection and testing devices and/or sample collection device collects exhalation from the nose and/or mouth. In some embodiments, the integrated sample collection and testing devices and/or sample collection device collects exhalation from the nose.

There is also a need for an inexpensive, simple to use, and reliable sample collection device that may be used by laypeople and/or medical professionals around the world to test to see if they are shedding pathogens or viruses. These sample collection devices may be paired with a testing device or method to determine the presence or absence of pathogens or viruses in the collected sample.

Some aspects of the present disclosure describe a sample collection device that includes a housing extending from a mouthpiece end to an air outlet end, the housing defining an airflow channel from the exhalation receipt or mouthpiece end to the air outlet end, the exhalation receipt or mouthpiece end configured to receive an exhalation airflow. A porous sample collection media is fixed within the housing and along the airflow channel. A exhalation receipt or mouthpiece end cap is replaceably coupled to the exhalation receipt or mouthpiece end and an air outlet end cap is replaceably coupled to the air outlet end.

The sample collection device may be combined with instructions for collecting a sample to form a kit. The instructions may instruct the user to: exhale into the airflow channel of the housing to capture a sample in the porous sample collection media: couple the air outlet end cap replaceably to the air outlet end to seal the air outlet end: flow a liquid through porous sample collection media: and couple the exhalation receipt or mouthpiece end cap to the exhalation receipt portion or mouthpiece end to seal the exhalation receipt portion or mouthpiece end. The sealed sample collection device may be transported to a testing location that is remote in time or location to the time and location the sample was collected.

In some embodiments, it may be desirable to provide a system that includes both a sample collector device and rapid testing device. The system may advantageously be self-contained and sterile (unlike swabs which may become contaminated upon use, pose biohazard risks during processing and opportunity for cross-contamination between samples). The system may also advantageously produce a cleaner sample for testing that is free to inhibitory substances, providing more accurate and reliable results.

A method of collecting a sample may include: flowing exhalation air into the sample collection device, forming a loaded porous sample collection media: sealing the air outlet end with the air outlet cap: flowing liquid through the loaded porous sample collection media, forming an eluent: and sealing the exhalation receipt portion or mouthpiece end with the exhalation receipt portion or mouthpiece end cap.

The eluent may be urged through a nozzle or liquid nozzle defining a dropper or liquid dropper fixed to the exhalation receipt portion or mouthpiece end cap or exhalation receipt portion or mouthpiece end. This expelled eluent may be tested using an assay such as a vertical or lateral assay.

### BRIEF DESCRIPTION OF FIGURES

FIG. 1 is a side elevation schematic diagram of an illustrative sample collection device collecting a sample.

FIG. 2 is a side elevation schematic diagram of an illustrative sample collection device flowing liquid through the porous sample collection media.

FIG. 3 is a side elevation schematic diagram of an illustrative sample collection device flowing liquid out of the porous sample collection media onto an assay.

### DEFINITIONS

All scientific and technical terms used herein have meanings commonly used in the art unless otherwise specified. The definitions provided herein are to facilitate understanding of certain terms used frequently herein and are not meant to limit the scope of the present disclosure.

The term “substantially” as used here has the same meaning as “significantly,” and can be understood to modify the term that follows by at least about 90%, at least about 95%, or at least about 98%. The term “not substantially” as used here has the same meaning as “not significantly,” and can be understood to have the inverse meaning of “substantially,” i.e., modifying the term that follows by not more than 10%, not more than 5%, or not more than 2%.

The term “about” is used here in conjunction with numeric values to include normal variations in measurements as expected by persons skilled in the art and is understood have the same meaning as “approximately” and to cover a typical margin of error, such as +5% of the stated value.

Terms such as “a,” “an,” and “the” are not intended to refer to only a singular entity but include the general class of which a specific example may be used for illustration.

The terms “a,” “an,” and “the” are used interchangeably with the term “at least one.” The phrases “at least one of” and “comprises at least one of” followed by a list refers to any one of the items in the list and any combination of two or more items in the list.

As used here, the term “or” is generally employed in its usual sense including “and/or” unless the content clearly dictates otherwise. The term “and/or” means one or all of the listed elements or a combination of any two or more of the listed elements.

The recitations of numerical ranges by endpoints include all numbers subsumed within that range (e.g., 1 to 5 includes 1, 1.5, 2, 2.75, 3, 3.80, 4, 5, etc, or 10 or less includes 10, 9.4, 7.6, 5, 4.3, 2.9, 1.62, 0.3, etc.). Where a range of values is “up to” or “at least” a particular value, that value is included within the range.

As used here. “have”. “having”. “include”. “including”. “comprise”. “comprising” or the like are used in their open-ended sense, and generally mean “including, but not limited to.” It will be understood that “consisting essentially of,” “consisting of,” and the like are subsumed in “comprising” and the like. As used herein. “consisting essentially of,” as it relates to a composition, product, method or the like, means that the components of the composition, product, method or the like are limited to the enumerated components and any other components that do not materially affect the basic and novel characteristic(s) of the composition, product, method or the like.

The words “preferred” and “preferably” refer to embodiments that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the disclosure, including the claims.

Any direction referred to here, such as “front.” “back.” “top.” “bottom.” “left.” “right.” “upper.” “lower.” and other directions and orientations are described herein for clarity in reference to the figures and are not to be limiting of an actual device or system or use of the device or system. Devices or systems as described herein may be used in a number of directions and orientations.

The terms “downstream” and “upstream” refer to a relative position based on a direction of exhalation airflow through the device. For example, the upstream-most element of the device is the mouthpiece element, and the downstream-most element of the device is the air outlet end.

### DETAILED DESCRIPTION

The present disclosure relates to sample collection devices, kits, systems, and methods. In some embodiments, the present disclosure relates to bioaerosol collection devices, kit, systems, and methods.

In some embodiments, a sample collection device includes a housing extending from a mouthpiece end to an air outlet end, the housing defining an airflow channel from the mouthpiece end to the air outlet end, the mouthpiece end configured to receive an exhalation airflow. A porous sample collection media is fixed within the housing and along the airflow channel. A mouthpiece end cap is replaceably coupled to the mouthpiece end and an air outlet end cap is replaceably coupled to the air outlet end.

The mouthpiece or exhalation receipt portion is configured to receive an exhalation airflow from one or more of the mouth or nose. For example, the mouthpiece or exhalation receipt portion can be breathed into by contact with or adjacency to the mouth or by contact with the nose/nostril or by contact with each individually or collectively. The exhalation airflow received by the mouthpiece or exhalation receipt portion can be one or both of oral exhalation or nasal exhalation. The term “mouthpiece.” where used herein, is meant to refer to an exhalation receipt portion that can receive oral or nasal exhalation of aerosol or bio-aerosol.

While the porous sample collection media is illustrated herein as defining a planar element, it is understood that the porous sample collection media may define any shape when disposed within the housing and along the airflow channel. For example, the sample collection media may be pleated. In some embodiments, the pleat frequency is between about 1 pleat per 0.6 cm of media and about 1 pleat per 2 mm of media. In some embodiments, the pleat height is between about 2 mm and about 4 mm.

The sample collection device includes a porous sample collection media disposed within the device housing and along an airflow channel defined by the device housing. Once a sample is collected on the porous sample collection media the open ends of the housing may be sealed for testing at a future time and/or remote location to the sample collection location. A liquid may be passed though the porous sample collection media after one or both open ends of the housing may be sealed.

One of the open ends of the housing may be closed or sealed with an end cap the includes a nozzle or liquid dropper. The nozzle or liquid dropper may further include a nozzle or dropper cap to seal the end of the nozzle or liquid dropper to maintain the liquid within the housing for testing at a future time and/or remote location to the sample collection location. The nozzle or liquid dropper provides a convenient way to locate drop-wise liquid from the housing onto an assay. Preferably a portion of the housing or one or both end caps may be “squeezable” to urge the liquid though the nozzle or liquid dropper and out of the sample collection device.

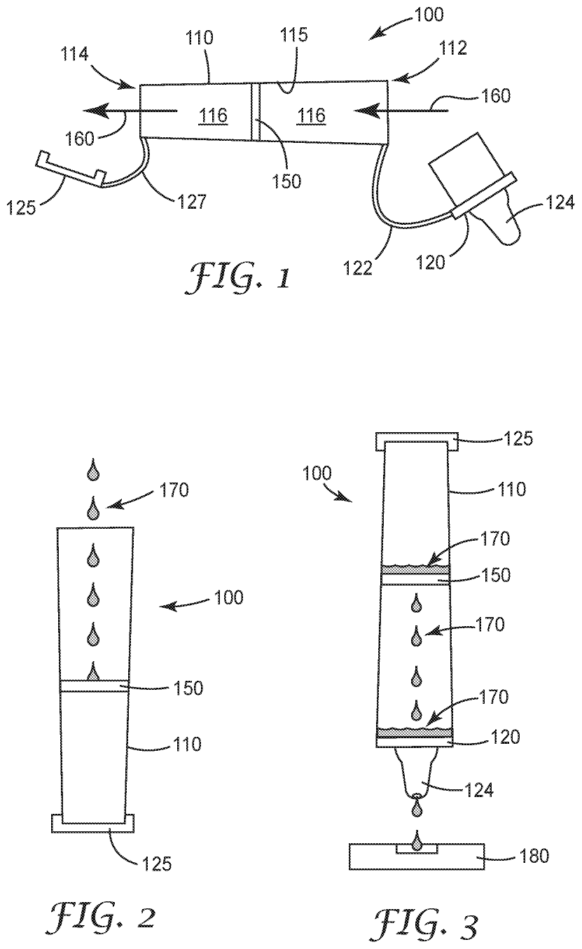

FIG. 1 is a side elevation schematic diagram of an illustrative sample collection device 100 collecting a sample. FIG. 2 is a side elevation schematic diagram of an illustrative sample collection device 100 flowing liquid 170 through the porous sample collection media 150. FIG. 3 is a side elevation schematic diagram of an illustrative sample collection device 100 flowing liquid 170 out of the porous sample collection media 150 onto an assay 180.

A sample collection device 100 includes a housing 110 extending from a mouthpiece end 112 to an air outlet end 114, the housing 110 defining an airflow channel 116 from the mouthpiece end 112 to the air outlet end 114, the mouthpiece end 112 is configured to receive an exhalation airflow 160 (eg: from the nose or mouth or both). A porous sample collection media 150 is fixed within the housing 110 and along the airflow channel 116. A mouthpiece end cap 120 is replaceably coupled to the mouthpiece end 112 and an air outlet end cap 125 is replaceably coupled to the air outlet end 114.

The housing 110 may define an open cylindrical airflow channel 116 extending along a longitudinal axis a length value L from the mouthpiece end 112 to the air outlet end 114. The open cylindrical airflow channel 116 may have a uniform diameter along the length value L from the mouthpiece end 112 to the air outlet end 114. The open cylindrical airflow channel 116 may have a decreasing diameter along the length value L tapering from the mouthpiece end 112 to the air outlet end 114. The open cylindrical airflow channel 116 may have an increasing diameter along the length value L tapering from the air outlet end 114 to the mouthpiece end 112.

The housing 110 is formed of a liquid and fluid impervious material. The housing 110 may be formed of a liquid and fluid impervious material that may be deformed or plasticly deformed by a user's finger pressure, similar to a squeeze bottle, to urge liquid out of the housing 110. The housing 110 may be formed of a polymer. The housing may have a uniform thickness in a range from 0.5 mm to 2 mm, for example. Advantageously, the user may squeeze or compress the housing 110 at the location of the porous sample collection media 150 to “wring out” liquid from the porous sample collection media 150.

The mouthpiece end cap 120 and the air outlet end cap 125 may be formed of the same type of material or different types of material. The mouthpiece end cap 120 and/or the air outlet end cap 125 may be formed of the same type of material as the housing 110. One or both of the mouthpiece end cap 120 and the air outlet end cap 125 may be squeezable, or be deformed or plasticly deformed by a user's finger pressure, similar to a squeeze bottle, to urge liquid out of the housing 110 (when both mouthpiece end cap 120 and the air outlet end cap 125 are attached to the housing 110). The mouthpiece end cap 120 and the air outlet end cap 125 may be formed of a polymer.

The mouthpiece end cap 120 and the air outlet end cap 125 may be separate from the housing 110. The mouthpiece end cap 120 may be coupled to the housing 110 via a tether 122 for example. The air outlet end cap 125 may be coupled to the housing 110 via a tether 127 for example. The tether 122 may be connected to any portion of the mouthpiece end cap 120. The tether 122 may be connected to any portion of the housing 110. The tether 127 may be connected to any portion of the air outlet end cap 125. The tether 127 may be connected to any portion of the housing 110.

One of the mouthpiece end cap 120 and the air outlet end cap 125 may further include a nozzle or liquid dropper 124. The nozzle or liquid dropper 124 may allow or be configured to allow the liquid to flow out of the housing 110 in a drop-wise manner, similar to an eye dropper. The nozzle or liquid dropper 124 may further include a nozzle or liquid dropper cap, configured to close or seal the nozzle or liquid dropper 124, similar to an eye-dropper cap. The nozzle or liquid dropper cap may be replaceably attached to the nozzle or liquid dropper 124 to preserve the liquid inside the sample collection device 100 for subsequent testing.

A liquid reservoir may be fluidly coupled to sample collection device 100 configured to provide a metered dose of liquid onto the porous sample collection media 150 is fixed within the housing 110. The metered dose of liquid is passed through the porous sample collection media 150 and carries away pathogen or virus that may be bound to the porous sample collection media 150. The metered dose of liquid 170 may then be analyzed.

The liquid reservoir may be fixed to the housing 110 and flow liquid through the sidewall of the housing 110. The liquid reservoir may be fixed to the mouthpiece end cap 120 and flow liquid through the wall of the mouthpiece end cap 120. The liquid reservoir may be fixed to the air outlet end cap 125 and flow liquid through the wall of the air outlet end cap 125.

Alternatively, a user may flow a metered dose of liquid onto the porous sample collection media 150 is fixed within the housing 110 by adding the liquid, dropwise for example, into one open end of the housing 110. In one example, the user may exhale through the open-ended housing 110, then seal the air outlet end 114 with the air outlet end cap 125, then add a metered dose of liquid 170 into the housing 110 via the open mouthpiece end 112, the metered dose of liquid 170 flows through the porous sample collection media 150 and strips away any biological material from the porous sample collection media 150, then the user seals the mouth end 112 with the mouth end cap 120. The user may then squeeze the sample collection device 100 to urge the liquid out of the housing 110, such as through a nozzle or liquid dropper 124, in a drop-wise manner onto an assay 180.

The metered dose of liquid 170 may be an aqueous liquid. The metered dose of liquid 170 may be an aqueous buffer solution. The metered dose of liquid 170 may be an aqueous liquid with a surfactant. The metered dose of liquid 170 may be saline solution. The metered dose of liquid 170 may be a saline solution comprising a surfactant. The metered dose of liquid 170 may be a saline solution comprising from 0.005% to 2% surfactant by weight. The metered dose of liquid 170 or liquid reservoir may have a volume in a range from 50 microliters to 500 microliters.

The porous sample collection media 150 may be fixed within the housing 110 and along the airflow channel 116. Exhalation airflow 160 passes through the thickness of the porous sample collection media 150. The sample collection device 100 is configured to allow a user to breath comfortably into the sample collection device 100 with minimal pressure drop across the porous sample collection media 150. For example, a user may exhale into the sample collection device 100 at a rate of less than 85 liters/min, or less than 50 liters/minute, or 35 liters/min, or less than 24 liters/min, or less than 10 liters/min and experience a pressure drop across the porous sample collection media 150 of 70 mm water or less, or 50 mm of water or less, or 25 mm or water or less, or 10 mm of water or less, or 5 mm of water or less.

The porous sample collection media 150 at least partially occludes the airflow channel 116. The porous sample collection media 150 may occlude the airflow channel 116. The porous sample collection media 150 may have a major plane that is orthogonal to the direction of the exhalation airflow 160 passing through the thickness of the porous sample collection media 150.

The porous sample collection media 150 is fixed within the airflow channel 116 and spaced apart from each of the air outlet end 114 to the mouthpiece end 112, to prevent a user from touching and contaminating the porous sample collection media 150. The housing 110 has a length value L from the mouthpiece end 112 to the air outlet end 114, and the porous sample collection media 150 is recessed from both the mouthpiece end 112 and the air outlet end 114 at least 20% of the length value L, or at least 25% of the length value L, or at least 30% of the length value L, or at least 35% of the length value L, or in a range from 25% to 50% of the length value L.

The porous sample collection media 150 may be a nonwoven material configured to filter or capture pathogens or virus from an exhalation airflow 160. The porous sample collection media 150 may be a nonwoven filtration layer or material having an electrostatic charge configured to filter or capture pathogens or virus from an exhalation airflow 160. The porous sample collection media 150 may be a hydrophobic nonwoven filtration layer or material configured to filter or capture pathogens or virus from an exhalation airflow 160. The porous sample collection media 150 may be a hydrophobic nonwoven filtration layer or material having an electrostatic charge configured to filter or capture pathogens or virus from an exhalation airflow 160.

The term “hydrophobic” refers to a material having a water contact angle of 90 degrees or greater, or from about 90 degrees to about 170 degrees, or from about 100 degrees to about 150 degrees. Water contact angle is measured using ASTM D5727-1997 Standard test method for surface wettability and absorbency of sheeted material using an automated contact angle tester.

The porous sample collection media 150 may be formed of polymeric material. The porous sample collection media 150 may be formed of a polyolefin. The porous sample collection media 150 may be formed of polypropylene. One illustrative porous sample collection media 150 is commercially available from 3M Company (St. Paul MN. U.S.A.) under the trade designation FILTRETE Smart MPR 1900 Premium Allergen. Bacteria & Virus Air Filter Merv 12 to 14, or Merv 13.

The porous sample collection media 150 may be formed of a of a polylactide (PLA) such as, for example. 6100D from Nature Works LLC15305 Minnetonka Blvd Minnetonka. MN 55345.

Exemplary nonwoven filtration layer or materials for use in or as the porous sample collection media 150 include, for example, those described in U.S. Pat. Nos. 7,947,142; 8,162,153: 9,139,940; and 10,273,612, all of which are incorporated herein in their entirety.

The porous sample collection media 150 may have a thickness (orthogonal to the major plane) in a range from 200 micrometers to 1000 micrometers, or from 250 micrometers to 750 micrometers. The porous sample collection media 150 may have major plane surface area in a range from about 1 cm2 to about 4 cm2, or about 2 cm2 to about 3 cm2, and is sufficient to fill the cross-section of the airflow channel 116. The airflow channel 116 may have a cross-sectional area in a range from 1 cm2 to about 4 cm2, or about 2 cm2 to about 3 cm2.

The porous sample collection media 150 defines a major surface area value and the liquid reservoir or metered dose of liquid 170 defines a volume value, and the volume value divided by the surface area value may be in a range from 10 microliters/cm2 to 400 microliter/cm2, or from 10 microliters/cm2 to 250 microliter/cm2, or from 50 microliters/cm2 to 150 microliter/cm2.

In some embodiments, the sample collection device 100 further includes a pre-filter or screen (not shown) disposed in the housing 110, and upstream of the porous sample collection media 150 to catch solid material or debris (such as, for example, food particles) so that they are not incident on the porous sample collection media 150. The pre-filter or screen (not shown) may be fixed within the housing 110 and along the airflow channel 116 and between the mouthpiece end 112 and the porous sample collection media 150. In some embodiments, the pre-filter or screen includes one or more flow apertures therethrough. The exhalation airflow passes through a thickness of the pre-filter or screen. The pre-filter or screen at least partially occludes the air flow channel 116. In some cases, the pre-filter or screen may have a major plane (not shown) that is orthogonal to the direction of the exhalation airflow (not shown) passing through the thickness of the pre-filter or screen. The pre-filter or screen may be a non-woven layer configured to filter out larger particles from the exhalation airflow passing through the pre-filter or screen. In some cases, the pre-filter or screen may be a non-woven layer that does not have an electrostatic charge. In some embodiments, the pre-filter or screen does not filter or capture significant amounts of viral or pathogen material and instead allows them to transmit through the pre-filter or screen. In some embodiments, the pre-filter or screen is made of or includes at least one of a plastic mesh, a woven net, a needle tacked fibrous web, a knitted mesh, an extruded net, and/or a carded or spunbond coverstock.

Two or more sample collection device 100 may be stacked end to end to apply the liquid onto two or more porous sample collection media 150 to test a group of samples with one assay 180.

The disclosure is also directed to a sample collection system. The sample collection system includes the sample collection device 100 described above and an assay 180 configured to receive liquid from the sample collection device 100. The assay 180 may be a separate element from the sample collection device 100. The assay 180 may be configured to receive liquid 170 from the sample collection device 100.

The assay 180 may be a flow assay, such as a lateral flow assay or a vertical flow assay. The assay 180 may detects a virus, pathogen, or other analyte of interest. The assay may be, for example, nucleic acid amplification (PCR, isothermal), immunoassays, etc. The assay 180 may include a test result display window to indicate the presence or absence of a virus or pathogen.

An assay may be referred to as lateral flow assays (LFAs) or vertical flow assays (VFAs) which are, generally, paper-based platforms for the detection and quantification of analytes in complex mixtures, where the sample is placed on a test device and the results are displayed within 5-30 min. Low development costs and ease of production of LFAs have resulted in the expansion of its applications to multiple fields in which rapid tests are required. LFA-based tests are widely used in hospitals, physician's offices and clinical laboratories for the qualitative and quantitative detection of specific antigens and antibodies, as well as products of gene amplification. A variety of biological samples can be tested using assays.

The disclosure is also directed to a kit including the sample collection device described above. and instructions for collecting a sample. The kit may be a package provided to a user to complete the sample collection process.

The kit instructions may include instructions to:

exhale into the airflow channel of the housing to capture a sample in the porous sample collection media:

couple the air outlet end cap replaceably to the air outlet end to seal the air outlet end;

flow a liquid through porous sample collection media: and

couple the mouthpiece end cap to the mouthpiece end to seal the mouthpiece end.

According to another embodiment, a method includes flowing exhalation air through a porous sample collection media. The porous sample collection media is disposed in an airflow channel forming a loaded porous sample collection media. Then flowing a metered dose of liquid through the loaded porous sample collection media disposed in the airflow channel forming an eluent and containing the eluent. Then the eluent may be tested with an assay.

The method may include flowing exhalation air into sample collection device described above, forming a loaded porous sample collection media, then sealing the air outlet end with the air outlet cap. then flowing liquid through the loaded porous sample collection media, forming an eluent, and then sealing the mouthpiece end with the mouthpiece end cap.

The method may further include, testing the eluent with an assay. The testing may include testing the eluent for a virus or pathogen presence. The method may include flowing a metered dose in a range from 50 microliters to 400 microliters of liquid through the loaded porous sample collection media disposed in the airflow channel. The method may include flowing a metered dose of aqueous liquid comprising a surfactant through the loaded porous sample collection media disposed in the airflow channel. The method may include squeezing the housing, air outlet end cap, or mouthpiece end cap to urge eluent through a nozzle or liquid dropper on the mouthpiece end cap.

All references and publications cited herein are expressly incorporated herein by reference in their entirety into this disclosure, except to the extent they may directly contradict this disclosure. Although specific embodiments have been illustrated and described herein, it will be appreciated by those of ordinary skill in the art that a variety of alternate and/or equivalent implementations can be substituted for the specific embodiments shown and described without departing from the scope of the present disclosure. It should be understood that this disclosure is not intended to be unduly limited by the illustrative embodiments and examples set forth herein and that such examples and embodiments are presented by way of example only with the scope of the disclosure intended to be limited only by the claims set forth here.

1. A sample collection device comprising:
a housing extending from a mouthpiece end to an air outlet end, the housing defining an airflow channel from the mouthpiece end to the air outlet end, the mouthpiece end configured to receive an exhalation airflow;
a porous sample collection media fixed within the housing and along the airflow channel;
a mouthpiece end cap replaceably coupled to the mouthpiece end; and
an air outlet end cap replaceably coupled to the air outlet end.

2. The sample collection device according to claim 1, wherein one of the mouthpiece end cap and the air outlet end cap, comprise a nozzle or liquid dropper.

3. The sample collection device according to claim 1, wherein one of the mouthpiece end cap or the air outlet end cap, comprise a liquid reservoir having a metered dose of liquid.

4. The sample collection device according to claim 3 wherein liquid reservoir defines a volume in a range from 50 microliters to 500 microliters.

5. The sample collection device according to claim 4, wherein the porous sample collection media defines a surface area value and the liquid reservoir defines a volume value, and the volume value divided by the surface area value is in a range from 10 microliters/cm2 to 400 microliter/cm2, or from 10 microliters/cm2 to 250 microliter/cm2.

6. The sample collection device according to claim 2, wherein the nozzle or liquid dropper further comprises a sealing cap configured to close-off the nozzle or liquid dropper.

7. The sample collection device according to claim 2, wherein the housing, mouthpiece end cap, or air outlet end cap are configured to be squeezed by a user to urge liquid out of the airflow channel and through the nozzle or liquid dropper.

8. The sample collection device according to claim 1, wherein the porous sample collection media comprises a nonwoven filtration layer having an electrostatic charge.

9. The sample collection device according to claim 8, wherein the nonwoven filtration layer is hydrophobic.

10. The sample collection device according to claim 8, wherein the nonwoven filtration layer is formed from at least one of polypropylene or polylactide.

11. The sample collection device according to claim 8, wherein the nonwoven filtration layer is pleated.

12. The sample collection device according to claim 8, wherein the nonwoven filtration layer has a thickness in a range from 200 to 1000 micrometers, or from 250 to 750 micrometers.

13. The sample collection device according to claim 3, wherein the liquid reservoir contains a liquid that is at least one of an aqueous fluid, an aqueous buffer solution, an aqueous fluid including a surfactant, a saline solution, or a saline solution including a surfactant.

14. The sample collection device according to claim 1, further comprising a pre-filter fixed within the housing and along the airflow channel and between the mouthpiece end and the porous sample collection media.

15. The sample collection device according to claim 1, wherein the housing has a length value L from the mouthpiece end to the air outlet end, and the porous sample collection media is recessed from both the mouthpiece end and the air outlet end at least 20% of the length value L.

16. The sample collection device according to claim 1, wherein the housing is formed of a polymer.

17. The sample collection device according to claim 1, wherein the mouthpiece end cap is tethered to the housing.

18. The sample collection device according to claim 1, wherein the air outlet end cap is tethered to the housing.

19. A sample collection system comprising:
the sample collection device of claim 1; and
an assay configured to receive liquid from the sample collection device.

20. The sample collection system according to claim 19, wherein the assay detects the presence of a virus, a pathogen, or other analytes of interest.

21-32. (canceled)
